(19) United States
(12) Patent Application Publication
(10) Pub. No.: US 2026/0174929 A1
(43) Pub. Date: Jun. 25, 2026

(54) IMPLANTABLE MEDICAL DEVICE HAVING MULTI-LAYER COATING

(71) Applicant: Pacesetter, Inc., Sylmar, CA (US)

(72) Inventor: Gopesh Tilvawala, Los Angeles, CA (US)

(21) Appl. No.: 19/427,825

(22) Filed: Dec. 19, 2025

Related U.S. Application Data
(60) Provisional application No. 63/738,445, filed on Dec. 23, 2024.

(51) Int. Cl. A61L 27/04; A61L 27/30; A61L 27/34; A61N 1/05

(52) U.S. Cl. CPC A61L 27/04; A61L 27/306; A61L 27/34; A61N 1/0587

(57) ABSTRACT

An implantable medical device includes a body and a coating. The body has a metal surface. The coating is deposited on the metal surface of the body. The coating includes a bonding layer and a polymer layer. The bonding layer includes a first layer comprising a first silane coupling agent and a second layer comprising a second silane coupling agent. The first layer is deposited on the metal surface, and the second layer is deposited on the first layer. The polymer layer is deposited on the second layer. The first silane coupling agent is capable of forming a stronger bond with the metal surface of the body than with the polymer layer. The second silane coupling agent is capable of forming a stronger bond with the polymer layer than the metal surface.

This application claims priority to U.S. patent application Ser. No. 63/738,445, entitled “implantable Medical Device Having Multi-layer Coating,” Filed Dec. 23, 2024, which is hereby incorporated by reference in its entirety.

### BACKGROUND

### 1. Technical Field

The present disclosure relates generally to coating for metallic substrates and, more specifically, coatings for implantable medical devices and methods of coating implantable medical devices with multi-layer coatings that resist delamination and extend the life of the implantable device.

### 2. Discussion of Related Art

Implantable medical devices, such as pacemakers or injection ports, often are implanted in patients for extended periods of time. These devices have coatings to improve their biocompatibility with the patient's body or for other functional purposes. However, the coatings can delaminate from the implantable device over time. Many implantable devices are made of metallic materials while many coatings are polymers. The differences in characteristics between the materials of implantable device and the coating can lead to delamination. This delamination can lead to failure of the implantable device and the need for replacement, which can introduce risk and complications that could otherwise be avoided.

As a more specific example, parylene-C is known in the field of implantable devices and often used as a coating for metallic medical devices for its excellent mechanical properties and biocompatibility. However, parylene-C coatings can delaminate from the metallic substrates with extended exposure to liquid environments.

### SUMMARY

Existing methods of addressing coating delamination include using a single-layer coating of a silane coupling agent, e.g., silane A-174, to attempt to improve the bond between the metallic surfaces of implantable devices and the parylene coating. Inclusion of silane A-174 may, however, decrease adhesion strength between the surface of the device and the parylene-C coating. A more robust bond between the metallic surfaces of implantable devices and the parylene coating is needed.

This disclosure relates generally to coatings for and methods of applying coatings to implantable medical devices. The present invention is defined, at least in part, in the independent claims. Further embodiments of the invention are defined in the dependent claims.

In an aspect of the present disclosure, an implantable medical device includes a body and a coating. The body has a metal surface. The coating is deposited on the metal surface of the body. The coating includes a bonding layer configured to form a gradient of interfacial bonding affinity between the metal surface and a polymer layer. The bonding layer includes a first layer comprising a first silane coupling agent and a second layer comprising a second silane coupling agent. The first layer is deposited on the metal surface, and the second layer is deposited on the first layer. The polymer layer is deposited on the second layer. The first silane coupling agent is capable of forming and/or configured to preferentially form a stronger bond with the metal surface of the body than with the polymer layer. The second silane coupling agent is capable of forming and/or configured to preferentially form a stronger bond with the polymer layer than the metal surface.

In aspects, the coating is configured to resist delamination from the metal surface when the implantable medical device is within a liquid environment. The polymer layer may include a parylene. The first layer may include the first silane coupling agent and a first dipodal. The second layer includes the second silane coupling agent and a second dipodal. The first layer and the second layer may each have a ratio of silane to dipodal in a range of 1:1 to 100:1.

In some aspects, the metal surface of the body is nano structured. The nano-structuring of the body strengthen mechanical bonding between the first layer and the metal surface. The nano-structuring may include several pores or fibers defined by the body of the implantable medical device such that the contact area between the first layer and the metal surface is increased.

In another aspect of the present disclosure, a biostimulator includes a housing and a coating. The housing includes a metal surface. The coating includes a bonding layer and a barrier layer. The bonding layer includes a first layer comprising a first silane coupling agent and a second layer comprising a second silane coupling agent. The barrier layer includes a polymer. The coating is configured to resist delamination from the metal surface of the biostimulator when the biostimulator is disposed within a liquid environment.

In aspects, the first layer includes the first silane coupling agent and a first dipodal. The first layer has a ratio of the first silane coupling agent to the first dipodal in a range of 1:1 to 100:1. The second layer includes the second silane coupling agent and a second dipodal. The second layer has a ration of the second silane coupling agent to the second dipodal in a range of 1:1 to 100:1. The polymer of the barrier layer may be a parylene. The coating may be configured to resist delamination when subjected to a pressure less than or equal to 650 kPa.

In another aspect of the present disclosure, a method includes depositing a bonding layer on a metal surface of an implantable medical device. The bonding layer includes a first layer comprising a first silane coupling agent and a second layer comprising a second silane coupling agent. The first layer is deposited on the metal surface, and the second layer is deposited on the first layer. The method also includes depositing a polymer layer on the second layer. The first silane coupling agent is capable of forming and/or configured to preferentially form a stronger bond with the metal surface than with the polymer layer. The second silane coupling agent is capable of forming and/or configured to preferentially form a stronger bond with the polymer layer than with the metal surface.

In aspects, the method also includes at least partially curing the first layer before depositing the second layer. Partially curing the first layer may include baking the implantable medical device at a temperature of less than or equal to 60 degrees Celsius for a duration in a range of 15 minutes to 25 minutes. The method may include at least partially curing the second layer before depositing the polymer layer.

In some aspects, depositing the first layer includes depositing the first silane coupling agent through a chemical vapor deposition process or a wet soaking process. Depositing the second layer may include depositing the second silane coupling agent through a chemical vapor deposition process.

In certain aspects, the first layer includes the first silane coupling agent and a first dipodal. The first layer may have a ratio of the first silane coupling agent to the first dipodal in a range of 1:1 to 100:1. The second layer may include the second silane coupling agent and a second dipodal. The second layer may have a ratio of the second silane coupling agent to the second dipodal in a range of 1:1 to 100:1. The method may also include nano-structuring the metal surface of the implantable medical device before depositing the first layer.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The various features of the present disclosure and the manner of attaining them will be described in greater detail with reference to the following description, claims, and drawings, wherein reference numerals are reused, where appropriate, to indicate a correspondence between the referenced items.

FIG. 1 is a front view of an example implantable medical device implanted in a patient, in accordance with an embodiment.

FIG. 2 is a side view another example implantable medical device, in accordance with an embodiment.

FIG. 3 is pictorial view of the example implantable medical device of FIG. 2 implanted within a heart, in accordance with an embodiment.

FIG. 4 is detail view of a coating deposited on an implantable medical device, in accordance with an embodiment.

FIG. 5 is a flowchart of a method of coating an implantable medical device, in accordance with an embodiment.

FIG. 6 is a detailed view of an implantable medical device prior to a nano-structuring process, in accordance with an embodiment.

FIG. 7 is a detailed view of an implantable medical device after a nano-structuring process, in accordance with an embodiment.

FIG. 8 is a detailed view of an implantable medical device after a nano-structuring process, in accordance with an embodiment.

### DETAILED DESCRIPTION

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

Many implantable devices are coated to improve the performance of the device within the body of a patient. However, conventional coatings for implantable devices and methods of applying coatings to implantable devices may yield an implantable device having an inadequate lifespan. Specifically, conventional coatings and methods can lead to delamination of the coating from the implantable device before the desired removal date. Delamination may be exacerbated when the implantable device is within a liquid environment, such as inside the human body. When the coating delaminates from the implantable device, the device may require replacement. This can lead to additional surgeries that could be avoided by a device with a longer lifespan. Often delamination is a result of poor bonding between two dissimilar materials. For example, many implantable devices are made of metallic material, e.g., titanium or stainless steel, while many coatings are polymers, e.g., parylene. These materials tend to exhibit different mechanical properties and tend to be unreactive with each other chemically. As such, the bonding between the devices and the coatings generally relies only on mechanical bonding, not chemical bonding, for adherence to one another. The difference in respective mechanical properties of the devices and the coatings can cause delamination, for example, from differences in thermal expansion and contraction, differences in modulus of elasticity, or differences in deflection under load. Accordingly, there is a need for improved coatings for implantable devices that resist delamination and extend the lifespan of implantable devices.

As described below, embodiments can include coatings for implantable medical devices, methods of coating implantable medical devices, and implantable medical devices including coatings that resist delamination and extend the lifespan of the device. Specifically, the coatings may include several layers that cooperate to increase adhesion strength between the coatings and the implantable medical devices. The coatings can significantly increase the lifespan of implantable medical devices when compared to conventional coatings.

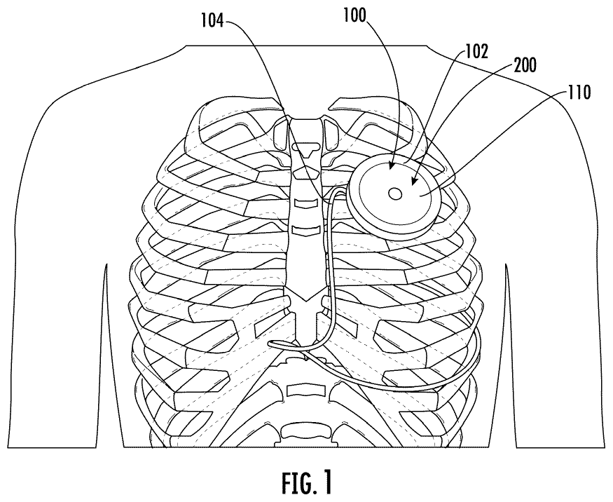

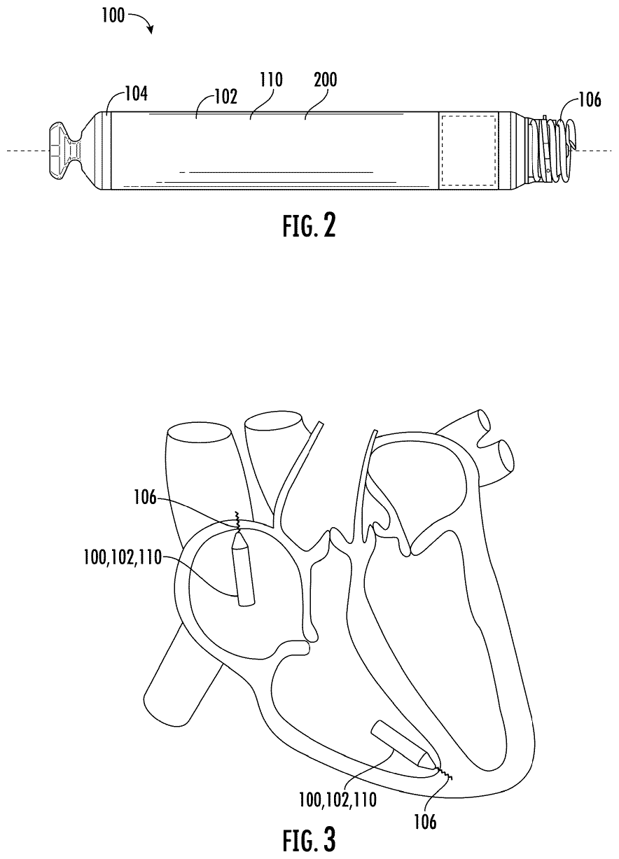

Referring now to FIGS. 1-3, examples of an implantable medical device (IMD) 100 in accordance with an embodiment are shown. The IMD 100 may be any number of implantable devices including, but not limited to, pacemakers, leadless pacemakers, implantable cardioverter defibrillators (ICD), pulse generators, implantable cardiac monitors, stents, ventricular assist devices, cochlear implants, artificial joints, implantable injection ports, implantable insulin pumps, or some other biostimulator, etc.

For example, in particular embodiments, where the IMD 100 is a biostimulator such as a pacemaker or a leadless pacemaker, the IMD 100 may include a housing 102 and one or more electrodes 104. For example, one electrode may be a shock electrode and another electrode may be a sensing electrode. More specifically, the electrodes 104 can deliver pacing pulses to muscle of a cardiac chamber, and optionally, can sense electrical activity from the muscle. The housing 102 can contain electronics such as a communications coil or antenna, a battery, or a pulse generator. The IMD 100 may communicate bidirectionally with at least one other device within or outside the body of the patient. The IMD 100 may be implanted within a subcutaneous pocket, below cutaneous tissue but above muscle tissue, as shown in FIG. 1. In some embodiments, the IMD 100 may be implanted submuscularly, e.g., below the pectoral muscles but above the ribcage. In certain embodiments, the IMD 100 may be implanted intracardially within a chamber of the heart, as shown in FIG. 3. In such embodiments, the IMD 100 may include one or more fixation elements 106. For example, the fixation element 106 may be a helical member to allow the IMD 100 to be screwed into the target tissue.

The IMDs can have a coating 200 bonded to a substrate or surface 110 of the body, e.g., the housing 102 of the IMD 100. In an embodiment, the coating 200 modifies the surface 110 of the IMD 100 to enhance biocompatibility and/or surface functionality of the IMD 100. More specifically, the performance of the materials of many IMDs 100 may be inadequate for implantation into the human body, where the IMD 100 will be exposed to tissues and bodily fluids. The coating 200 modifies the surface 110 of the IMD 100 to better interface with body tissues and body fluids. For example, the coating 200 may reduce friction between body tissues and the IMD 100, and may fluidly seal the IMD 100, or may reduce an inflammatory response to implantation of the IMD 100 into a patient. In some embodiments, the coating 200 may electrically insulate the IMD 100. Additionally, the coating 200 resists delamination from the IMD 100 and extends the lifespan of the IMD 100. More specifically, the coating 200 both mechanically and chemically bonds to the surface 110 to resist delamination. Bonding the coating 200 to the IMD 100 both mechanically and chemically may extend the lifespan of the IMD 100 when in a fluid environment. More particularly, the coating 200 may experience pressure less than or equal to 1,000 kilopascals (kPa) and remain adhered to the surface 110. The coating 200 may experience and resist delamination under such pressures in a dry or a liquid environment.

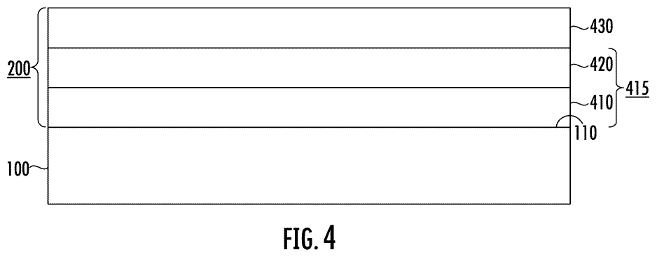

Referring to FIG. 4, a detailed view of the coating 200 on the IMD 100 is shown in accordance with an embodiment. The coating 200 includes a first layer 410, a second layer 420, and a third layer 430. In some embodiments, the coating 200 may include more than three layers. The third layer 430, or the outermost layer, can be a polymer layer or a biomaterial layer. The third layer 430 may be a barrier layer with low permeability to liquids. As used herein the term “biomaterial” refers to material that are configured to interface with the human body. The biomaterial may be a biocompatible material and/or a bioactive material. The biomaterial may be a metallic material, a polymer, or a ceramic, etc. The first layer 410 and the second layer 420 are disposed between the third layer 430 and the surface 110 of the IMD 100. The first layer 410 and the second layer 420 cooperate to establish a graded chemical and mechanical transition region between the surface 110, e.g., a metal surface, and the third layer 430, e.g., a polymer layer. The cooperation can bond the third layer 430 to the IMD 100. The first layer 410 and second layer 420 together may be considered a bonding layer 415 to bond the third layer 430 to the surface 110. In general, the coating 200 is configured to bond two dissimilar materials to each other. For example, the surface 110 may be a metal surface, the third layer 430 may be a polymer layer, and the bonding layer 415 may be disposed between the surface 110 and the third layer 430.

The first layer 410, the second layer 420, and the third layer 430 may each have a thickness in a range of 10 nanometers (nm) to 1 micrometer (μm), e.g., 100 nm, 250 nm, 300 nm, 500 nm, or 750 nm. Each of the first layer 410, the second layer 420, and the third layer 430 may have an equal thickness. In embodiments, each of the layers 410, 420, 430 may have different thicknesses. In some embodiments, the thickness of the coating 200, i.e., the sum of the thickness of the first layer 410, the second layer 420, and the third layer 430, can be less than or equal to 1 μm. For example, each of the first layer 410, the second layer 420, and the third layer 430 may have equal thicknesses of 300 nm that sum to a coating thickness of 900 nm. In certain embodiments, the first layer 410 and the second layer 420 are thinner than the third layer 430. In such an embodiment, the first layer 410 and the second layer 420 may each have a thickness of 250 nm and the third layer 430 may have a thickness of 500 nm such that the coating 200 has a thickness of 1 μm. In particular embodiments, the thickness of the coating 200 may be greater than 1 μm, e.g., 1.5 μm, 2 μm, 2.5 μm, or 3 μm.

The first layer 410 is deposited on the surface 110 of the IMD 100. The first layer 410 mechanically and chemically bonds to the surface 110. More specifically, the first layer 410 includes a first coupling agent that bonds well with the material of the surface 110. For example, the first coupling agent may form a strong bond such as a covalent bond or an ionic bond with the surface 110, as opposed to a weak bond like a hydrogen bond. In embodiments, the first coupling agent may form multiple covalent bonds, e.g., double bonds or triple bonds, with the material of surface 110. The first coupling agent may be capable of forming and/or configured to preferentially form a stronger bond with the surface 110 than with the third layer 430. For example, the first coupling agent may be capable of forming more than one single, covalent bond per molecule with the surface 110, as in the case of a trialkoxysilane. In contrast, the first coupling agent may be capable of forming one single, covalent bond per molecule with the third layer 430, as in the case of a monoalkoxysilane. In some embodiments, the first coupling agent may be capable of forming one or more double or triple covalent bonds with the surface 110 and only single covalent bonds with the third layer 430. In embodiments, where the surface 110 is made of titanium the coupling agent may include a silane that bonds well to titanium. More particularly, the silane of the first coupling agent may have a similar hydrolytic stability to that of the material of the surface 110, e.g., titanium or titanium oxide. The similarity of hydrolytic stability between the silane and the surface 110 may encourage bonding. In embodiments, the silane may negate or ignore an inert oxide layer of the surface 110 or alcohol groups of reactants. In general terms, the silane of the first coupling agent may have a the formula R(CH₂)ₙSiX₃ where R is a non-hydrolyzable organic radical that may possess a functionality that imparts a desired characteristic to the coating 200 and X is a hydrolyzable group including, but not limited to, alkoxy, acyloxy, halogen, or amine groups. In one non-limiting example, where the surface 110 of the IMD 100 to be coated is a titanium surface, the first coupling agent may include the silane STYRYLETHYLTRIMETHOXYSILANE (C₁₃H₂₀O₃Si), sold by Gelest® as SIS6990.0. The silane may form strong bonds with an inorganic material, like metals and metal oxides, e.g., titanium or titanium oxide.

In some embodiments, the first coupling agent may include a dipodal. Dipodals can improve the hydrolytic stability of the bond between the silane of the first coupling agent and the surface 110 of the IMD 100. Improved hydrolytic stability can prevent cured polymers, e.g., the first layer 410, from reverting to a semisolid or liquid form when exposed to high humidity or liquid environments, e.g., within the human body. Specifically, the dipodal of the first coupling agent may be 1,8-BIS(TRIMETHOXYSILYL)OCTANE (C₁₄H₃₄O₆Si₂) sold by Gelest® as SIB1832.7. More generally, the dipodal may include two silicone atoms that can covalently bond with the surface 110. The availability of the two silicone atoms to bond with the surface 110 may significantly increase adhesion strength between the first layer 410 and the surface 110. The first coupling agent may have a ratio of silane to dipodal in a range of 100:1 to 1:1, e.g., a silane to dipodal ratio of 10:1. The first coupling agent may consist essentially of a silane or may consist essentially of a silane and a dipodal. In alternative embodiments, the coupling agents may comprise other organofunctional silanes, multi-alkoxysilanes, or hybrid organometallic coupling agents exhibiting preferential bonding characteristics. For example, the first coupling agent may additionally include elements that do not alter the function of the first coupling agent of bonding with the surface 110 or the second layer 420. For example, dyes may be added to the first coupling agent to change the color of the first layer 410. Changing the color of the first layer 410 may aid in controlling layer thickness during manufacture by providing clear visual delineation between the first layer 410 and the surface 110. In some embodiments, the emulsifiers or thinners may be added to the first coupling agent to alter its viscosity. Controlling the viscosity of the first coupling agent may aid in uniform application of the first layer 410. For example, a more viscous first coupling agent may allow for a uniform and relatively thick first layer 410. In an embodiment, the bonding layer is configured such that chemical affinity to the metal surface decreases with distance from the surface 110, while chemical affinity to the polymer layer increases with distance from the surface 110.

In embodiments where the first coupling agent includes both a silane and a dipodal, the first layer 410 may have an increased number of bonding sites with the surface 110 of the IMD 100. Specifically, inclusion of dipodal in the first coupling agent can double the number of bonding sites with the surface 110 and, thus, increases hydrolytic stability. Where the first coupling agent includes a dipodal the silane has six hydrolyzable groups, e.g., three bonds on each of the two silicone molecules of the dipodal, that can bond with the material of the surface 110. In contrast, where the first coupling agent excludes a dipodal the silane may have three hydrolyzable groups (e.g., trialkoxysilane) or one hyrdolyzable group (e.g., monoalkoxysilane) available for bonding. In such an embodiment, the energy required to dissociate the bond between the surface 110 and the first layer 410 including a first coupling agent excluding dipodal may be in a range of 410 kJ/mol to 585 kJ/mol. Inclusion of dipodal in the first coupling agent can substantially increase resistance to hydrolytic degradation under prolonged exposure to physiological liquid environments. For example, the inclusion can increase resistance to hydrolysis by 100,000 times when compared to a first coupling agent excluding dipodal. This increased resistance to hydrolysis can significantly reduce the instances of delamination and, thus, extend the lifespan of the IMD 100. As a result, the lifespan for bonds between the first layer 410 and the surface 110 including dipodal may extend functional adhesion lifetime by orders of magnitude relative to single-layer coupling systems. For example, the lifespan can be extended by 10,000 months for every 1 month of lifespan for bonds without dipodal. Accordingly, the lifespan for the coating 200, and by extension the IMD 100, can be extended by the same degree.

Continuing to refer to FIG. 4, the second layer 420 is deposited on IMD 100 on top of the first layer 410. The second layer 420 bonds to the first layer 410. The second layer 420 includes a second coupling agent that bonds well with the material of third layer 430. For example, the second coupling agent may form a strong bond such a covalent bond or an ionic bond with the third layer 430, as opposed to a weak bond like a hydrogen bond. In embodiments, the first coupling agent may form multiple covalent bonds, e.g., double bonds or triple bonds, with the material of third layer 430. The second coupling agent may be capable of forming and/or configured to preferentially form a stronger bond with the third layer 430 than with surface 110. For example, the second coupling agent may be capable of forming more than one single, covalent bond per molecule with the third layer 430, as in the case of a trialkoxysilane. In contrast, the second coupling agent may be capable of forming one single, covalent bond per molecule with the third layer 430, as in the case of a monoalkoxysilane. In some embodiments, the second coupling agent may be capable of forming one or more double or triple covalent bonds with the third layer 430 and only single covalent bonds with the surface 110. In embodiments, where the third layer 430 is a parylene, e.g., parylene-C, the second coupling agent may include a silane that bonds well to the selected parylene. More particularly, the silane of the second coupling agent may have a similar hydrolytic stability as that of the material of the third layer 430, e.g., a parylene layer. The similarity of hydrolytic stability between the silane and the third layer 430 may encourage bonding. In general terms, the silane of the second coupling agent may have a the formula R(CH₂)ₙSiX₃ where R is a non-hyrdolyzable organic radical that may possess a functionality that imparts a desired characteristic to the coating 200 and X is a hydrolyzable group including, but not limited to, alkoxy, acyloxy, halogen, or amine groups. In one example embodiment, the second coupling agent may include the silane ((chloromethyl)phenylethyl)trimethoxysilane (C₁₂H₁₉Clo₃Si), sold by Gelest® as SIC2295.5. In some embodiments, the silane of the second coupling agent may be the same as the silane of the first coupling agent of the first layer 410. Silanes readily bond with one another. As such, the silane of the second layer 420 can bond with the silane of the first layer 410. In embodiments, the energy required to dissociate the bond between the first layer 410 and the second layer 420 may be in a range of 290 kJ/mol to 505 kJ/mol. Accordingly, there is a reduced likelihood that the coating 200 will experience delamination between the first layer 410 and the second layer 420.

In embodiments, the second coupling agent may include a dipodal. The dipodal of the second coupling agent may be the same as the dipodal of the first coupling agent, e.g., 1,8-BIS(TRIMETHOXYSILYL)OCTANE (C₁₄H₃₄O₆Si₂) sold by Gelest® as SIB1832.7. The second coupling agent may have a ratio of silane to dipodal in a range of 100:1 to 1:1, e.g., a silane to dipodal ratio of 10:1. The second coupling agent may consist essentially of a silane or may consist essentially of a silane and a dipodal. For example, the second coupling agent may additionally include elements that do not alter the function of the second coupling agent of bonding with the first layer 410 or the third layer 430.

In embodiments where the second coupling agent includes both a silane and a dipodal, the second layer 420 may have an increased number of bonding sites with the third layer 430. Inclusion of dipodal may also increase the number of bonding sites with the first layer 410. Specifically, inclusion of dipodal in the second coupling agent can double the number of bonding sites with the third layer 430 or the first layer 410. As described above, including a dipodal in the second coupling agent increases the number hydrolyzable groups of the silane available for bonding to six. In contrast, where the second coupling agent excludes a dipodal the silane has three hydrolyzable groups available for bonding. In such an embodiment, the energy required to dissociate the bond between the third layer 430 and the second layer 420 including a second coupling agent excluding dipodal may be in a range of 335 kJ/mol to 700 kJ/mol. Inclusion of dipodal in the second coupling agent may materially reduce hydrolysis-driven bond cleavage at the bonding interfaces. For example, the inclusion can increase resistance to hydrolysis by 100,000 times when compared to a second coupling agent excluding dipodal. This increased resistance to hydrolysis can reduce the instances of delamination. As a result, the lifespan for bonds between the second layer 420 and the first layer 410 or the third layer 430 including dipodal may be extended by 10,000 months for every 1 month of lifespan for bonds without dipodal.

Continuing to refer to FIG. 4, the third layer 430 is deposited on the IMD 100 on top of the second layer 420. The third layer 430 bonds to the second layer 420, as described above. The third layer 430 may be a biomaterial configured to interface with body tissue and body fluids when the IMD 100 is implanted within a patient. The third layer 430 may interface with the internal structures and fluids of a human body, e.g., tissues such as bone, muscle, fascia, or adipose tissue and fluids such as blood. More specifically, the third layer 430 may be configured to achieve a particular biological response from the human body. For example, the third layer 430 may illicit little to no immune response from the human body as a result of the implantation of the IMD 100. For example, in such an embodiment, the third layer 430 may be a parylene. In embodiments, the third layer 430 may be a parylene-C (C₁₆H₁₄C₁₂), a parylene-N (C₁₆H₁₆), or a parylene-F (C₁₈H₈F₈). Parylene is a biomaterial that is non-toxic and physiologically nonreactive. In embodiments, the third layer 430 may include or consist essentially of a parylene. For example, the third layer 430 may additionally include elements that do not alter bonding with the second layer 420 or the biomaterial function of the third layer 430. For example, dyes may be added to the first coupling agent to change the color of the third layer 430. Changing the color of the third layer 430 may aid in controlling layer thickness during manufacture by providing clear visual delineation between the third layer 430 and the second layer 420. In some embodiments, the emulsifiers or thinners may be added to the first coupling agent to alter its viscosity. Controlling the viscosity of the first coupling agent may aid in uniform application of the third layer 430. For example, a more viscous first coupling agent may allow for a uniform and relatively thick third layer 430. Parylene can be applied in thin layers, e.g., less than 1 μm thick, and conforms well to surface features. These aspects make parylene coatings desirable for coating the IMD 100. In some embodiments, the third layer 430 may be any inert polymers that may be applied via chemical vapor deposition. For example, the third layer 430 may be Teflon® (PTFE) or polyimide.

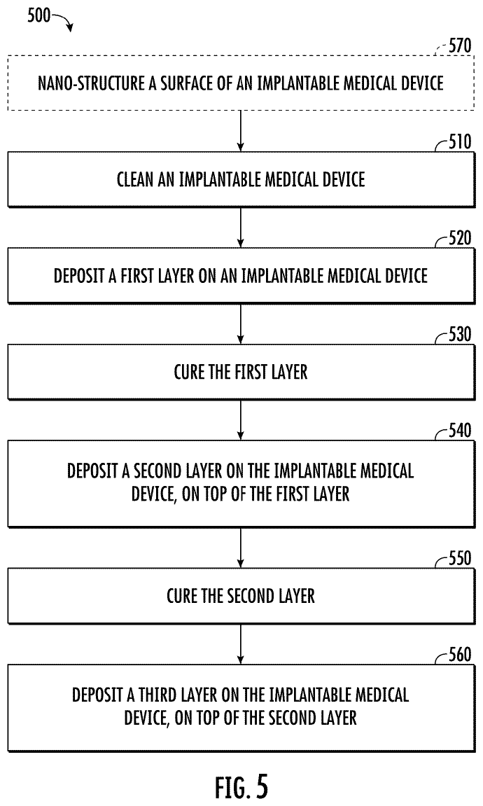

Referring to FIG. 5, a method 500 of coating an implantable medical device in accordance with an embodiment is described with reference to the example IMDs 100 and the coating 200 of FIGS. 1-4. It will be appreciated that method 500 is provided by way of example, and the operations described may be added to or subtracted from, including being performed in different orders, to manufacture the structures described above.

At operation 510 the IMD 100 is cleaned. Cleaning the IMD 100 may remove debris and/or impurities, e.g., dust or lubricants from manufacturing, from the surface 110 of the IMD 100. The IMD 100 may be cleaned with any reagent conventionally used to clean implantable devices. For example, the reagent may be, but is not limited to, isopropyl alcohol (C₃H₈O), ethanol (C₂H₆O), acetone (C₃H₆O), or combinations thereof. The IMD 100 may be submerged within the reagent and cleaned within a stirred bath, a wet bath, or an ultrasonic bath.

In embodiments, cleaning at operation 510 may include drying the IMD 100 after removal from the bath. Drying the IMD 100 removes any moisture or reagent remaining from submerging the IMD 100 in the bath. The IMD 100 may be vacuum baked to dry the remaining moisture from the surface 110 of the IMD 100. Specifically, the IMD 100 may be placed in a vacuum oven and baked at a temperature in a range of 40 degrees Celsius to 60 degrees Celsius, e.g., 50 degrees Celsius. In particular embodiments, the IMD 100 may be baked in a range of 100 degrees Celsius to 200 degrees Celsius, e.g., 110, 125, 150, or 175 degrees Celsius. The IMD 100 may be baked at higher temperatures when there are no temperature sensitive electronics contained within the IMD 100. The IMD 100 may be baked in the vacuum oven for a duration in the range of 20 minutes to 24 hours, e.g., 30 minutes, 1 hour, 4 hours 12 hours, or 18 hours.

Optionally, cleaning at operation 510 may include plasma cleaning after drying the IMD 100. Electrical components with the IMD 100 may be damaged when subjected to plasma cleaning. Accordingly, in embodiments where the IMD 100 includes electrical components at the cleaning operation 510, additional plasma cleaning at operation 510 may be foregone. Plasma cleaning of the IMD 100 may be performed by any conventional plasma cleaning processes. For example, the plasma cleaning process may use oxygen, nitrogen, argon, hydrogen, or a combination thereof, as the gases for ionization. In embodiments, plasma cleaning may be performed for a duration in a range of less than 1 minute to 30 minutes, e.g., 15 minutes and at a power in a range of 1 Watt to 300 Watts, e.g., 150 Watts.

Continuing to refer to FIG. 5, at operation 520 the first layer 410 is deposited on to the surface 110 of the IMD 100. The first layer 410 includes the first coupling agent which may be configured to bond to the surface 110 of the IMD 100. The first coupling agent may mechanically and/or chemically bond with the surface 110 to adhere the first layer 410 thereto. The first coupling agent can improve the adherence between the surface 110 of the IMD 100 and another material that may bond poorly with the surface 110 itself, e.g., the second layer 420 or the third layer 430. The first coupling agent may be a silane. Specifically, the first coupling agent may include a silane that bonds well to the material of the IMD 100. For example, where the surface 110 of the IMD 100 to be coated with the coating 200 is made of titanium the silane of the first coupling agent may be SIC6990.0 from Gelest®. In some embodiments, the first coupling agent may include a dipodal. Mixing a silane and a dipodal to form the first coupling agent may increase the number of bonding sites between the first layer 410 and the surface 110 of the IMD 100, as described above. In embodiments, the dipodal of the first coupling agent may be SIB1832.7 from Gelest®. The first coupling agent may be mixed to have a ratio of silane to dipodal in a range of 100:1 to 1:1, e.g., a silane to dipodal ratio of 10:1.

The first layer 410 may be applied to the surface 110 of the IMD 100 by a wet soak process or a chemical vapor deposition (CVD) process. When the first layer 410 is applied in a wet soak process the IMD 100 is submerged in a soak solution containing the first coupling agent. The soak solution may include water, a silane solvent, and the first coupling agent, e.g., an amount of silane and dipodal with a ratio of 10:1. The silane solvent may be, but is not limited to, isopropyl alcohol (C₃H₈O), methanol (CH₃OH), ethanol (C₂H₆O), or a combination thereof. The soak solution may have a concentration of water less than or equal to 60% of the total volume of the soak solution. The soak solution may be stirred to mix the first coupling agent, the water, and the silane solvent to a homogenous solution. The ratio of the first coupling agent to the silane solvent may be in a range of 1:1 to 10:1, e.g., 5:1.

Once the soak solution is homogeneously mixed, the IMD 100 is submerged in the soak solution for deposition of the first layer 410 on to the surface 110 of the IMD 100. The soak solution may be continuously stirred to circulate the soak solution about the IMD 100 and to maintain homogeneity of the soak solution. The IMD 100 may be submerged for a duration greater than or equal to 15 minutes, e.g., 15 minutes, 20 minutes, 30 minutes, 45 minutes, 1 hour, 3 hours, or more than 3 hours. The duration of the wet soak process may be dictated by the desired thickness of the first layer 410. The wet soak process may deposit the first layer 410 with a thickness in a range of 10 nm to 1 μm, e.g., 50 nm, 100 nm, 500 nm, or 800 nm. In particular embodiments, the thickness of the first layer 410 may be greater than 1 μm. In some embodiments, the wet soak process may be performed in a pressurized system to decrease the duration the IMD 100 is submerged in the soak solution. For example, pressurizing the wet soaking process to a pressure in the range of 5 kilopascals (kPa) to 100 kPa, e.g., 50 kPa, may decrease the duration the IMD 100 is submerged in the soak solution to a duration less than or equal to 5-minutes.

In some embodiments, the first layer 410 is deposited by a CVD process. The CVD process may be any chemical vapor deposition process known in the art. More specifically, when the first layer 410 is deposited by CVD the IMD 100 may be placed in a vacuum chamber. The first coupling agent may be introduced to the vacuum chamber and deposited on the surface 110 of the IMD 100. The first coupling agent may be introduced to the vacuum chamber as a premixed solution, e.g., a mixture having a ratio of 10:1 silane to dipodal. In some embodiments, the constituent elements of the first coupling agent may be introduced to the vacuum chamber separately and mixed within the vacuum chamber or in-line to the vacuum chamber. The CVD process may last for a duration of minutes to hours depending on the desired thickness of the first layer 410. For example, the CVD process may have duration of 5 minutes, 10 minutes, 15 minutes, 30 minutes, 45 minutes, 1 hour, 2 hours, 3 hours or longer than 3 hours. The CVD process may deposit the first layer 410 with a thickness in a range of 10 nanometers (nm) to 1 micrometer (μm), e.g., 50 nm, 100 nm, 500 nm, or 800 nm. In particular embodiments, the first layer 410 is deposited with a thickness of more than 1 μm.

Continuing to refer to FIG. 5, at operation 530, the first layer 410 may be partially cured or fully cured. To cure the first layer 410 the IMD 100 is baked in an oven or vacuum oven. Curing in a vacuum oven may remove bubbles from within the first layer 410 and encourage a uniform thickness. Whether the first layer 410 is partially cured or fully cured depends on the duration and the temperature at which the IMD 100 is baked. The IMD 100 may be baked at a temperature less than or equal to 70 degrees Celsius, e.g., 60 degrees Celsius. In some embodiments, the temperature and duration of the cure may be determined based on the absence or presence of electronics within the IMD 100. When electronics are present, a lower baking temperature for a longer bake time may reduce the likelihood of damage to electronics. The duration that the IMD 100 is baked to cure the first layer 410 may vary from minutes to hours. For example, the IMD 100 may be baked for 5 minutes, 10 minutes, 15 minutes, 30 minutes, 45 minutes, 1 hour, 2 hours, 3 hours, 6 hours, 12 hours, 24 hours, or more than 24 hours. In particular embodiments, where the first layer 410 is partially cured, the IMD 100 may be baked for a duration of 20 minutes at a temperature of 55 degrees Celsius. In certain embodiments, where first layer 410 is fully cured, the IMD 100 may be baked for a duration of 1 hour at a temperature of 55 degrees Celsius. Partial curing of the first layer 410 prior to deposition of the second layer 420 can enable interdiffusion and covalent interlinking across the layer interface. Accordingly, partially curing the first layer 410 may improve the bonding between the first layer 410 and the second layer 420. Specifically, depositing the second layer 420, as described below, on top of a partially cured first layer 410 may provide for more binding sites between the first layer 410 and the second layer 420. Additionally or alternatively, partially curing the first layer 410 may allow for the first layer 410 and the second layer 420 to comingle at the interface between them and increase the strength of mechanical bonding therebetween.

At operation 540, the second layer 420 is deposited on the IMD 100 on top of the first layer 410. The second layer 420 includes the second coupling agent. The second coupling agent of the second layer 420 may be different from the first coupling agent of the first layer 410. The second coupling agent may be configured to bond well to the first layer 410, e.g., the silane of the first coupling agent, and/or the material of the third layer 430. The second coupling agent may resist delamination between the layers 410, 420, and 430 of the coating 200. The second coupling agent may include a silane. Specifically, the second coupling agent may include a silane that bonds well to the material of the third layer 430. For example, where the third layer 430 is a parylene material the silane of the second coupling agent may be SIC2295.5 from Gelest®. More specifically, silanes generally bond well to each other, e.g., the silane of the first coupling agent and the silane of the second coupling agent, and the silane of the second coupling agent may bond well to the third layer 430. Accordingly, the second coupling agent cooperates with the first coupling agent to adhere the third layer 430 to the IMD 100.

In some embodiments, the second coupling agent may include a dipodal. Mixing a silane and a dipodal to form the second coupling agent may increase the number of bonding sites between the second layer 420 and the third layer 430 or the second layer 420 and the first layer 410. The dipodal of the second coupling agent may be the same dipodal of the first coupling agent. For example, the dipodal of the second coupling agent may be SIB1832.7 from Gelest®. In some embodiments, the dipodal of the second coupling agent may be a different dipodal than that of the first coupling agent. The second coupling agent may have a ratio of silane to dipodal in a range of 100:1 to 1:1, e.g., a silane to dipodal ratio of 10:1.

The second layer 420 may be deposited on the IMD 100 by a CVD process. More specifically, the second layer 420 is deposited by a deposition process selected to reduce the likelihood of solvent-induced disruption, swelling, or dissolution of the first layer 410 after partial curing. The process may not expose the first layer 410 to a solvent that would dissolve the first layer 410 from the surface 110 of the IMD 100. For example, where the first layer 410 contains a silane the second layer 420 is deposited without exposing the first layer 410 to a silane solvent such as water or isopropyl alcohol. The CVD process to deposit the second layer 420 may be any CVD process known in the art. For example, where the second layer 420 is deposited by CVD the IMD 100 may be placed in a vacuum chamber, with the partially cured or fully cured first layer 410 deposited on the surface 110. The second coupling agent may be introduced to the vacuum chamber and deposited on the surface 110 of the IMD 100 on top of the first layer 410. The second coupling agent may be introduced to the vacuum chamber as a premixed solution. In some embodiments, the constituent elements of the second coupling agent may be introduced to the vacuum chamber separately and mixed within the vacuum chamber or in-line to the vacuum chamber. The CVD process may last for a duration of minutes to hours depending on the desired thickness of the second layer 420. For example, the CVD process may have a duration of 5 minutes, 10, minutes, 15, minutes, 30 minutes, 45 minutes, 1 hour, 2 hours, 3 hours or longer than 3 hours. The CVD process may deposit the second layer 420 with a thickness in a range of 10 nanometers (nm) to 1 micrometer (μm), e.g., the 50 nm, 100 nm, 500 nm, or 800 nm. In particular embodiments, the second layer 420 may be deposited with a thickness greater than 1 μm.

Continuing to refer to FIG. 5, at operation 550, the second layer 420 is partially cured or fully cured. To cure the second layer 420 the IMD 100 is baked in an oven or vacuum oven. Curing in a vacuum oven may remove bubbles from within the second layer 420 and encourage a uniform thickness. Whether the second layer 420 is partially cured or fully cured depends on the duration and the temperature at which the IMD 100 is baked. The IMD 100 may be baked at a temperature less than or equal to 70 degrees Celsius, e.g., 60 degrees Celsius. The duration that the IMD 100 is baked to cure the second layer 420 may vary from minutes to hours. For example, the IMD 100 may be baked for 5 minutes, 10 minutes, 15 minutes, 30 minutes, 45 minutes, 1 hour, 2 hours, 3 hours, or more than 3 hours. In particular embodiments, where the second layer 420 is partially cured, the IMD 100 may be baked for a duration of 20 minutes at a temperature of 55 degrees Celsius. In certain embodiments, where the second layer 420 is fully cured, the IMD 100 may be baked for a duration of 1 hour at a temperature of 55 degrees Celsius. Partial curing of the second layer 420 can establish reactive sites that promote covalent attachment of the polymer layer without solvent-induced degradation of underlying layers. Accordingly, partially curing the second layer 420 may improve the bonding between the second layer 420 and the third layer 430. Specifically, depositing the third layer 430, as described below, on top of a partially cured second layer 420 may provide for more binding sites between the second layer 420 and the third layer 430. Additionally or alternatively, partially curing the second layer 420 may allow for the second layer 420 and the third layer 430 to comingle at the interface between them and increase the strength of mechanical bonding therebetween.

At operation 560, a third layer 430 of a biomaterial is deposited on the IMD 100 on top of the second layer 420. The third layer 430 may interface with the internal structures and fluids of a human body, e.g., tissues such as bone, muscle, fascia, or adipose tissue and fluids such as blood. More specifically, the third layer 430 may be configured to achieve a particular biological response from the human body. For example, the third layer 430 may illicit little to no immune response from the human body as a result of the implantation of the IMD 100. In such an embodiment, the third layer 430 may be a parylene material. For example, the third layer 430 may be parylene-C, parylene-N, or parylene-F.

The third layer 430 is deposited without exposing the bonding layer to solvents capable of re-solubilizing or chemically modifying silane-based coupling structures. Accordingly, deposition can occur without exposing the IMD 100 to a solvent that will dissolve the first layer 410 or the second layer 420. For example, where the first layer 410 and the second layer 420 contain a silane the third layer 430 may be deposited without exposing the first layer 410 or the second layer 420 to a silane solvent such as water or isopropyl alcohol. As such, the third layer 430 may be deposited by a CVD process. The CVD process to deposit the second layer 420 may be any chemical vapor deposition process known in the art. More specifically, where the third layer 430 is deposited by CVD the IMD 100 may be placed in a vacuum chamber, including the partially cured or fully cured first layer 410 and the partially cured or fully cured second layer 420. The material of third layer 430 may be introduced to the vacuum chamber and deposited on the surface 110 of the IMD 100 on top of the second layer 420. The CVD process may last for a duration of minutes to hours depending on the desired thickness of the third layer 430. For example, the CVD process may have a duration of 5 minutes, 10, minutes, 15, minutes, 30 minutes, 45 minutes, 1 hour, 2 hours, 3 hours or longer than 3 hours. The CVD process may deposit the third layer 430 with a thickness in a range of 10 nm to 1 μm, e.g., 50 nm, 100 nm, 500 nm, or 800 nm. In particular embodiments, the third layer 430 is deposited with a thickness greater than 1 μm.

In embodiments, the third layer 430 may be cured. For example, where the third layer 430 is a parylene material, the third layer 430 may be cured by any method known in the art for curing parylene. For example, curing the third layer 430 may include baking the IMD 100. The third layer 430 is cured without exposing the first layer 410 or the second layer 420 to a solvent that may damage the layers 410, 420. Damage to the first layer 410 or the second layer 420 may interfere with the adherence of the third layer 430. In embodiments, the third layer 430 may be cured without exposing the third layer 430 to an overly humid environment. Humidity may cause damage or contaminate the third layer 430. In some embodiments, where the first layer 410 or the second layer 420 are partially cured, curing the third layer 430 can include simultaneously fully curing the first layer 410 or the second layer 420.

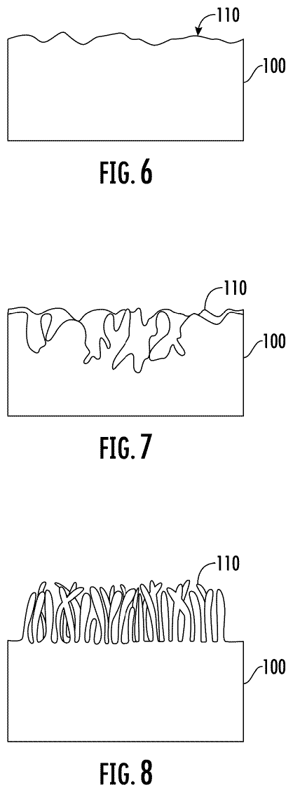

Additionally referring to FIGS. 6-8, in particular embodiments, optionally at operation 570, the IMD 100 is subjected to nano-structuring processes. Nano-structuring the surface 110 of the IMD 100 increases the surface area of the IMD 100 and can create chemically active anchoring sites that cooperate with the first coupling agent to form mechanically interlocked and chemically bonded interfaces. The nano-structuring may improve mechanical bonding of the first layer 410 thereto. As shown in FIG. 6, prior to nano-structuring, the surface 110 of the IMD 100 may be substantially smooth and non-porous. Nano-structuring the surface 110 of the IMD 100 may define an interconnected network of pores (FIG. 7) or fibers (FIG. 8) extending a depth into the surface 110 of the IMD 100. The nanostructures may extend a depth into the surface 110 in a range of 1 μm to 5 μm, e.g., 1.5 μm or 2 μm. The pores or the fibers may have a diameter in a range of 5 nm to 50 nm, e.g., 20 nm, 25 nm, or 30 nm. When the first layer 410 is deposited onto the surface 110 a portion of the first layer 410 may fill the pores or the space between the fibers. The increased contact area between the first layer 410 and the nano-structured surface 110 may increase adhesion strength between the first layer 410 and the surface 110 of the IMD 100. The surface 110 after nano-structuring may have a specific surface area (SSA) that is 250 times larger than prior to nano-structuring. In some embodiments, the nano-structuring process may oxidize the surface 110. For example, where the surface 110 is a titanium material, the nano-structuring process may increase the presence of titanium oxide (TiO₂). As noted above, the first coupling agent may include a silane that bonds well with inorganic material like metal oxides.

The IMD 100 may be nano-structured before cleaning the IMD 100 at operation 510. In embodiments, the nano-structuring process may be a soaking process that includes immersing the IMD 100 in a caustic solution, e.g., a sodium hydroxide (NaOH) solution. The caustic solution may be a 1 Mole/Liter (M/L) to 10 M/L, e.g., 5 M/L, aqueous solution of NaOH. In some embodiments, the soaking process may be performed in any other appropriate solvent that increases the surface area of the surface 110. For example, the soak solution may be acidic, e.g., a solution of hydrochloric acid. The caustic solution may be heated and stirred for the duration of the soak. For example, the soak solution may be heated to a temperature in a range of 40 degrees Celsius to 80 degrees Celsius, e.g., 60 degrees Celsius for a duration in a range of 12 hours to 48 hours, e.g., 24 hours. Additionally or alternatively, the IMD 100 may be laser machined, e.g., with a femtosecond laser, to increase the effective surface area of the surface 110. The nano-structured surface enhances silane condensation reactions by increasing hydroxyl group density.

In some embodiments, the nano-structuring process may be a hydrothermal process that includes submerging the IMD 100 in a caustic solution, e.g., a 1 M/L aqueous solution of NaOH, and heating the IMD 100 while submerged in the caustic solution in an autoclave at a temperature in a range of 50 degrees Celsius to 200 degrees Celsius, e.g., 180 degrees Celsius, for a duration in a range of 1 hour to 10 hours, e.g., 2 hours or 6 hours. After heating, the IMD 100 may be allowed to cool within the autoclave or at room temperature within ambient air. The IMD 100 may be cooled for a predetermined period of time in a range of 1 hour to 24 hours, e.g., 2 hours. In embodiments, the IMD 100 may be allowed to cool until a desired temperature is reached, e.g., room temperature.

In certain embodiments, the nano-structuring process may be an ethylene glycol enhanced hydrothermal process that includes submerging the IMD 100 in a caustic solution of NaOH and ethylene glycol (C₂H₆O₂). For example, the caustic solution may be a mixture of a 3 M/L aqueous NaOH solution with a 1:1 solution of ethylene glycol and water. The IMD 100 may be heated within an autoclave while submerged in the caustic solution at a temperature in a range of 50 degrees Celsius to 230 degrees Celsius, e.g., 200 degrees Celsius, for a duration in a range of 1 hour to 10 hours, e.g., 2 hours or 6 hours. After heating, the IMD 100 may be allowed to cool within autoclave or at room temperature. The IMD 100 may be cooled for a predetermined period of time in a range of 1 hour to 24 hours, e.g., 2 hours. In embodiments, the IMD 100 may be allowed to cool until a desired temperature is reached, e.g., room temperature.

Continuing to refer to FIGS. 5-8, each nano-structuring process described above may yield different nanostructures on the surface 110 of the IMD 100. For example, the soak process may yield a larger number of pores than fibers and the hydrothermal process may yield a greater number of fibers than pores. Altering individual parameters, e.g., temperatures, durations, or solution concentrations, of the nano-structuring process may alter the resulting nanostructures.

After the IMD 100 is subjected to one of the above-described nano-structuring processes, the IMD 100 may be washed in a neutralizing solution to neutralize any caustic solution remaining on the IMD 100. For example, where the IMD 100 is immersed in a NaOH solution, and alkaline solution, the neutralizing solution may be an acidic solution, e.g., a hydrochloric acid (HCl) solution. In particular embodiments, the neutralizing solution may be a 0.5 mM/L aqueous HCl solution. The IMD 100 may be immersed in the neutralizing solution for a duration of minutes, e.g., 5, 10, or 15 minutes, to several hours, e.g., 12, 16, 20, or 24 hours. The neutralization solution may be made maintained at an elevated temperature for the duration of neutralization. The temperature of the neutralization solution may be in a range of 30 degrees Celsius to 60 degrees Celsius, e.g., 40 degrees Celsius.

In embodiments, the nano-structuring operation 570 may include drying the IMD 100. The IMD 100 may be dried in an oven, a convention oven, or a vacuum oven. For example, the IMD 100 may be dried at a temperature in a range of 20 degrees Celsius to 60 degrees Celsius, e.g., 40 degrees Celsius, for a duration in a range of 0.5 hours to 4 hours, e.g., 2 hours.

The nano-structuring operation 570 may include calcinating the IMD 100. The IMD 100 may be calcinated at a temperature in a range of 300 degrees Celsius to 600 degrees Celsius, e.g., 400 degrees Celsius, for a duration in a range of 0.5 hours to 2 hours, e.g., 1 hour. The heating rate during calcination may be in a range of 1° C./min to 10°C./min, e.g., 5° C./min. The IMD 100 may be calcinated in air, nitrogen, or a vacuum. The furnace used for calcination may be cooled naturally to room temperature before removing the IMD 100 therefrom.

Although the method operations or steps are described in a specific order, it should be understood that other operations and steps may be performed in between described operations and steps, described operations and steps may be adjusted so that they occur at slightly different times, or the described operations and steps may occur in any order unless otherwise specified.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. An implantable medical device, comprising:
a body having a metal surface; and
a coating deposited on the metal surface of the body, the coating comprising:
a bonding layer including a first layer comprising a first silane coupling agent and a second layer comprising a second silane coupling agent, the first layer deposited on the metal surface of the body and the second layer deposited on the first layer; and
a polymer layer deposited on the second layer, wherein the first silane coupling agent is capable of forming a stronger bond with the metal surface of the body than with the polymer layer, and wherein the second silane coupling agent is capable of forming a stronger bond with the polymer layer than the metal surface.

2. The implantable medical device according to claim 1, wherein the coating is configured to resist delamination from the metal surface when the implantable medical device is within a liquid environment.

3. The implantable medical device according to claim 2, wherein the polymer layer comprises a parylene.

4. The implantable medical device according to claim 3, wherein the first layer includes the first silane coupling agent and a first dipodal, and wherein the second layer includes the second silane coupling agent and a second dipodal, the first layer and the second layer each having a ratio of silane to dipodal in a range of 1:1 to 100:1.

5. The implantable medical device according to claim 1, wherein the metal surface of the body is nano-structured to strengthen mechanical bonding between the first layer and the metal surface.

6. The implantable medical device according to claim 5, wherein the nano-structuring comprises a plurality of pores or fiber defined by the body of the implantable medical device such that a contact area between the first layer and the metal surface is increased.

7. A biostimulator, the biostimulator comprising:
a housing including a metal surface; and
a coating deposited on the metal surface of the housing, the coating comprising:
a bonding layer including a first layer comprising a first silane coupling agent and a second layer comprising a second silane coupling agent; and
a barrier layer comprising a polymer, wherein the coating is configured to resist delamination from the metal surface of the biostimulator when the biostimulator is disposed within a liquid environment.

8. The biostimulator according to claim 7, wherein the first layer includes the first silane coupling agent and a first dipodal, the first layer having a ratio of the first silane coupling agent to the first dipodal in a range of 1:1 to 100:1.

9. The biostimulator according to claim 7, wherein the second layer includes the second silane coupling agent and a second dipodal, the second layer having a ratio of the second silane coupling agent to the second dipodal in a range of 1:1 to 100:1.

10. The biostimulator according to claim 7, wherein the polymer of the barrier layer is a parylene.

11. The biostimulator according to claim 7, wherein the coating is configured to resist delamination when subjected to a pressure less than or equal to 650 kPa.

12. A method, comprising:
depositing a bonding layer on a metal surface of an implantable medical device, the bonding layer including a first layer comprising a first silane coupling agent and a second layer comprising a second silane coupling agent, the first layer deposited on the metal surface and the second layer deposited on the first layer; and
depositing a polymer layer on the second layer, wherein the first silane coupling agent is capable of forming a stronger bond with the metal surface than with the polymer layer, and wherein the second silane coupling agent is capable of forming a stronger bond with the polymer layer than with the metal surface.

13. The method according to claim 12, further comprising at least partially curing the first layer before depositing the second layer.

14. The method according to claim 13, wherein partially curing the first layer includes baking the implantable medical device at a temperature of less than or equal to 60 degrees Celsius for a duration in a range of 15 minutes to 25 minutes.

15. The method according to claim 12, further comprising at least partially curing the second layer before depositing the polymer layer.

16. The method according to claim 12, wherein depositing the first layer includes depositing the first silane coupling agent through a chemical vapor deposition process or a wet soaking process.

17. The method according to claim 16, wherein depositing the second layer includes depositing the second silane coupling agent through a chemical vapor deposition process.

18. The method according to claim 12, wherein the first layer includes the first silane coupling agent and a first dipodal, the first layer having a ratio of the first silane coupling agent to the first dipodal in a range of 1:1 to 100:1.

19. The method according to claim 12, wherein the second layer includes the second silane coupling agent and a second dipodal, the second layer having a ratio of the second silane coupling agent to the second dipodal in a range of 1:1 to 100:1.

20. The method according to claim 12, further comprising nano-structuring the metal surface of the implantable medical device before depositing the first layer.
